(19) United States
(12) Patent Application Publication
(10) Pub. No.: US 2003/0065327 A1
(43) Pub. Date: Apr. 3, 2003

(54) Biopolar ablation electrodes and method of use

(75) Inventors: Parris S. Wellman, Hillsborough, NJ (US); Rajesh Pendekanti, Bridgewater, NJ (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: ETHICON, INC., Somerville, NJ (US)

(21) Appl. No.: 10/027,134

(22) Filed: Dec. 20, 2001

Related U.S. Application Data
(63) Continuation-in-part of application No. 09/966,765, filed on Sep. 28, 2001.
(63) Continuation-in-part of application No. 09/966,813, filed on Sep. 28, 2001.

(51) Int. Cl. A61B 18/18

(52) U.S. Cl. 606/51

(57) ABSTRACT

Ablation instruments and methods for ablating tissue, and more particularly for treating atrial fibrillation utilizing RF energy are provided. The ablation instrument generally includes two components: a first member adapted to be placed on or adjacent to a first tissue surface, and a second member opposed to the first member and adapted to be placed on or adjacent to a second, opposed tissue surface. Each member includes a conductive element disposed on a portion thereof that is effective to communicate with a source of ablative energy. First and second conductor elements can be provided for transmitting ablative energy from an energy source to the first and second conductive elements. In use, ablative radiation is transmitted between the first and second members through the intervening tissue to form a lesion in the tissue.

### CROSS REFERENCE TO RELATED APPLICATIONS

This application is a continuation-in-part of U.S. patent application Ser. No. 09/966,756, filed Sep. 28, 2001, entitled “SURGICAL TREATMENT FOR ATRIAL FIBRILLATION USING RADIOFREQUENCY TECHNOLOGY,” and U.S. patent application Ser. No. 09/966,813, filed Sep. 28, 2001, entitled “TRANSMURAL ABLATION TOOL AND METHOD.”

### FIELD OF THE INVENTION

The present invention relates to instruments and methods for ablating tissue, and more particularly to surgical instruments and methods for ablating cardiac tissue using radiofrequency energy.

### BACKGROUND OF THE INVENTION

Cardiac arrhythmias, such as atrial fibrillation, are a commonly occurring disorder characterized by erratic beating of the heart. The regular pumping function of the atria is replaced by a disorganized, ineffective quivering caused by chaotic conduction of electrical signals through the upper chambers of the heart. While medication can be an effective treatment for some cases, many patients are not responsive to medical therapies and require alternative treatment. As an alternative to medication, a surgical technique, known as the Maze technique, requires open chest surgery to strategically incise the atrial wall, and subsequently repair the incisions by suturing. The result of this surgery is to create scar tissue located along the incision lines and extending through the atrial wall to block electrical conductivity from one segment to another.

While the Maze procedure has proven effective in restoring normal sinus rhythm, it requires considerable prolongation of cardiopulmonary bypass and aortic crossclamp time, especially when performed in combination with other open heart procedures. Over the last decade, more simplified techniques have been proposed which replace surgical incisions with ablations, or scars, formed in the heart tissue. The various energy sources used in ablation technologies include cryogenic, radiofrequency (RF), laser, and microwave energy. The ablation devices are used to create tissue lesions in an affected portion of the heart in order to block electrical conduction.

One common ablation technique employs the use of a catheter that is introduced into the heart (e.g., intravascularly) to direct RF energy at specific areas of heart tissue found to be the source of the irregular rhythms. An electrophysiology (EP) study is first performed to discover the location and characteristics of the arrhythmia and, once the specific location is identified and mapped, RF energy is delivered to the tissue to ablate the tissue, thus forming a lesion that blocks electrical conduction. While minimally invasive techniques are usually preferred, the procedure is often performed in combination with other open heart procedures as a prophylactic to prevent post-operative onset of atrial fibrillation.

RF ablation techniques are typically successful in treating atrial fibrillation, however the lesions must be well defined within the heart to be effective. The lesion must have a sufficient length, continuity and/or depth to interrupt or to block electrical conduction across the affected portion of the heart. This can be difficult to achieve without forming an incision in the atrium. In addition, if the energy is not uniformly transmitted to the target site, hot spots can form, possibly leading to severe tissue damage or blood coagulation (clots).

Accordingly, there exists a need for ablation instruments and procedures that produce uniform ablations with minimal risk of damage to the atria.

### SUMMARY OF THE INVENTION

The present invention provides ablation instruments and methods for ablating tissue, and more particularly for treating atrial fibrillation utilizing RF energy. The ablation instrument generally includes two components: a first member adapted to be placed on or adjacent to a first tissue surface, and a second member opposed to the first member and adapted to be placed on or adjacent to a second, opposed tissue surface. The members are preferably elongate members, and can be malleable. Each member includes a conductive element disposed on a portion thereof that is effective to communicate with a source of ablative energy. First and second conductor elements can be provided for transmitting ablative energy from the energy source to the first and second conductive elements. In use, ablative radiation is transmitted between the first and second members through the intervening tissue to form a lesion in the tissue.

In one embodiment, the first and second members are movable relative to each other at least between a first, open position, and a second, closed position in which the first member is adjacent to the second member. An actuating member can be mated to the first and/or second member to selectively move the members between the open and closed positions. The actuating member can include, for example, opposed first and second handles, wherein a force applied to bring the first and second handles in contact with each other causes opening of the first and second members. Conversely, a force applied to separate the first and second handles from each other causes the first and second members to close. The first and second members can optionally be biased to one of the open and closed positions.

In other aspects, the first and second members can be elongate members having a proximal end mated to the actuating member and a distal end having the conductive element disposed thereon. An insulative coating can be disposed around a portion, preferably the distal portion, of at least one of the first and second members. In an exemplary embodiment, the distal portion of the second member includes a tissue piercing tip adapted to be selectively deployed into tissue.

In another embodiment, the first conductive element is formed from first and second electrodes extending along the length of the distal portion of the first member. The electrodes are adapted to be positioned adjacent a tissue surface. The second conductive element is formed from a single electrode extending along the length of the distal portion of the second member. The single electrode is adapted to be positioned adjacent an opposed tissue surface between the first and second electrodes of the first member.

In other aspects, one of the first and second conductive elements is an active, energy transmitting electrode, and the other one of the first and second conductive elements is a return electrode. The energy transmitting electrode is effective to transmit ablative radiation between intervening tissue and the return electrode to form a lesion in the tissue. Preferably the surface area of the return electrode is greater than the surface area of the active energy transmitting electrode.

Methods of ablating tissue are also provided.

### BRIEF DESCRIPTION OF THE DRAWINGS

The invention will be more fully understood from the following detailed description taken in conjunction with the accompanying drawings, in which:

FIG. 1 is a schematic illustration of a surgical ablation instrument having first and second members positioned in a closed position according to one embodiment of the present invention;

FIG. 2 is an illustration of the surgical ablation instrument of FIG. 1 having the first and second members positioned in an open position;

FIG. 3 is a schematic illustration of another embodiment of a surgical ablation instrument according to the present invention;

FIG. 4A is a cross-sectional view of a distal portion of one embodiment of the first and second members of a surgical ablation instrument according to the present invention;

FIG. 4B is a cross-sectional view of a distal portion of another embodiment of the first and second members of a surgical ablation instrument;

FIG. 4C is a cross-sectional view of a distal portion of yet another embodiment of the first and second members a surgical ablation instrument;

FIG. 5A is a schematic representation of an ablation instrument having a first member positioned on an epicardial surface of a heart and a second member positioned on an endocardial surface of the heart; and

FIG. 5B is an illustration of an ablation instrument having first and second members positioned on opposed surfaces of tissue.

### DETAILED DESCRIPTION OF THE INVENTION

The present invention provides surgical ablation instruments and methods for ablating tissue, and more particularly for treating atrial fibrillation. The instrument and methods are particularly effective to form a lesion uniformly through the entire thickness of the tissue, e.g. the myocardial wall. The methods can be performed during open-heart surgical procedures, through an open incision, or using minimally invasive techniques. Moreover, the methods can be performed on either a beating heart or an arrested heart. The techniques according to the present invention offer more control and precision in treating conditions such as atrial fibrillation.

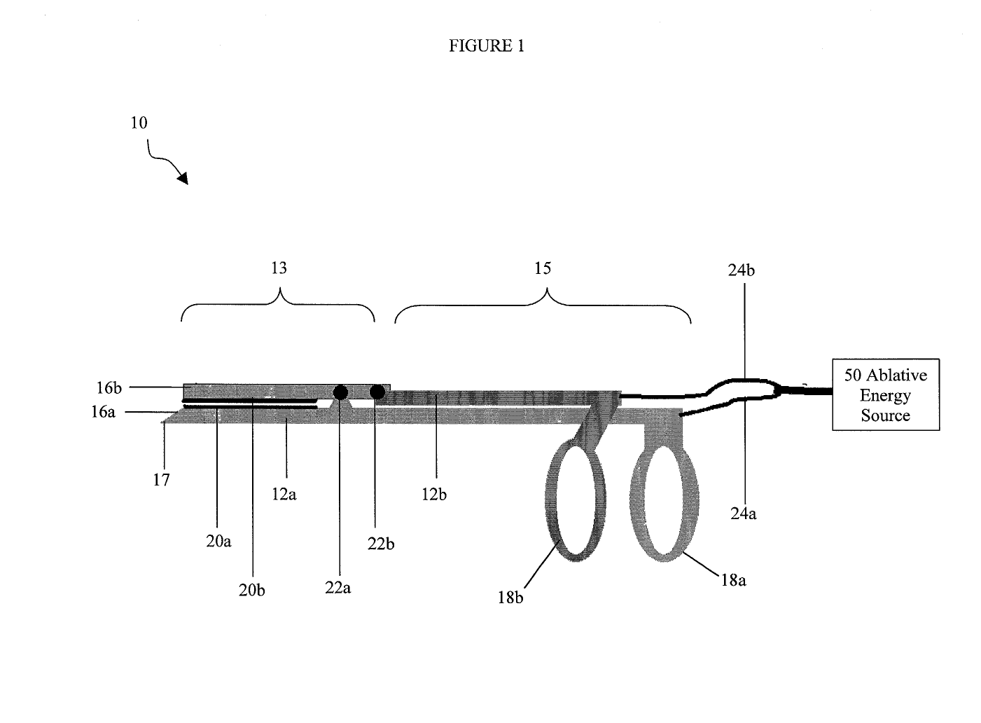

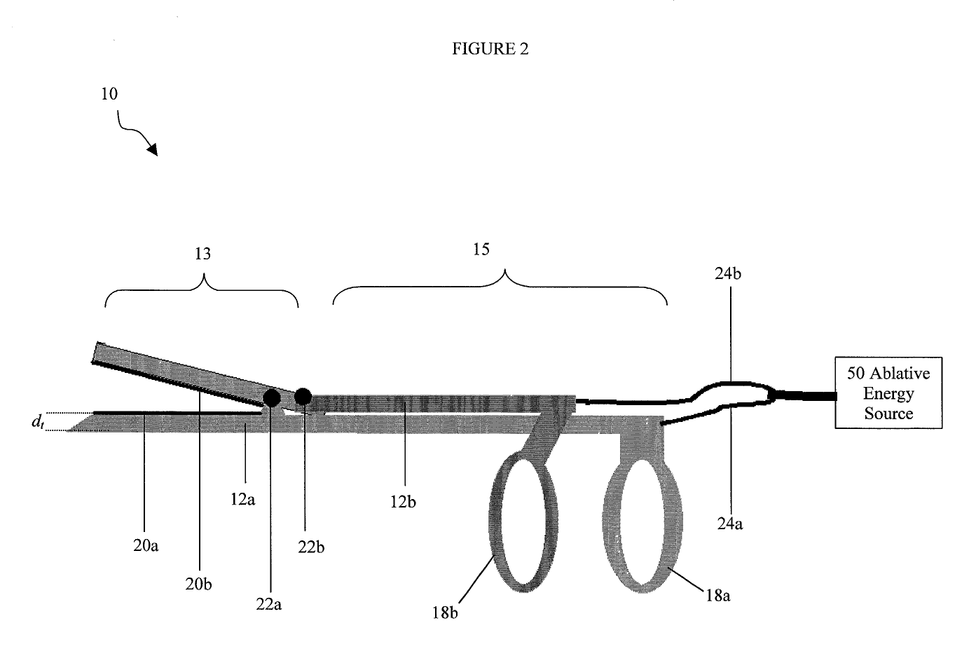

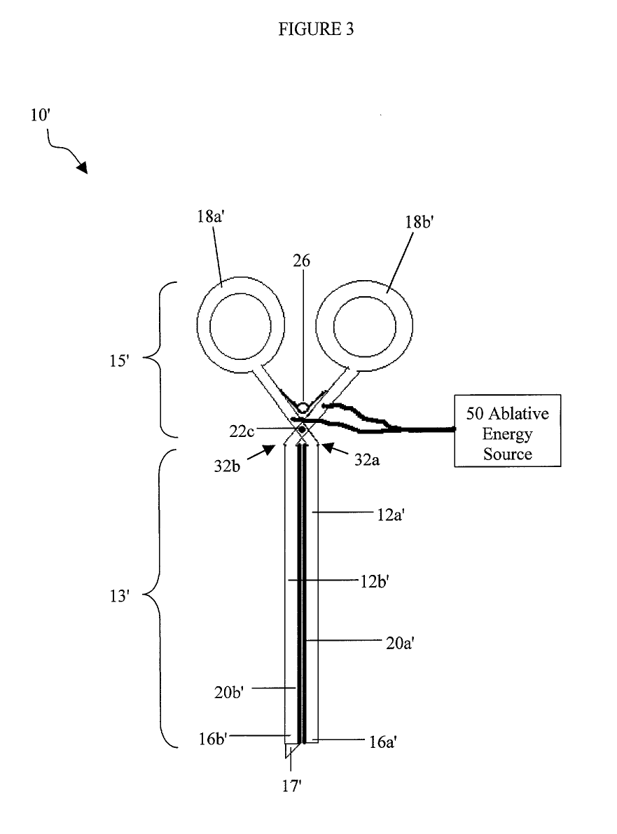

As shown in FIGS. 1-3, the surgical ablation instrument 10, 10′ generally includes first and second members 12a, 12b, 12a′, 12b′ movably mated to each other and including a proximal portion 15, 15′ and distal portion 13, 13′. The first and second members 12a, 12b, 12a′, 12b′ are preferably mated to each other at the proximal portion 15, 15′ of the instrument 10, 10′, and are movable at least between a first, open position, as shown in FIG. 2, and a second, closed position, as shown in FIGS. 1 and 3.

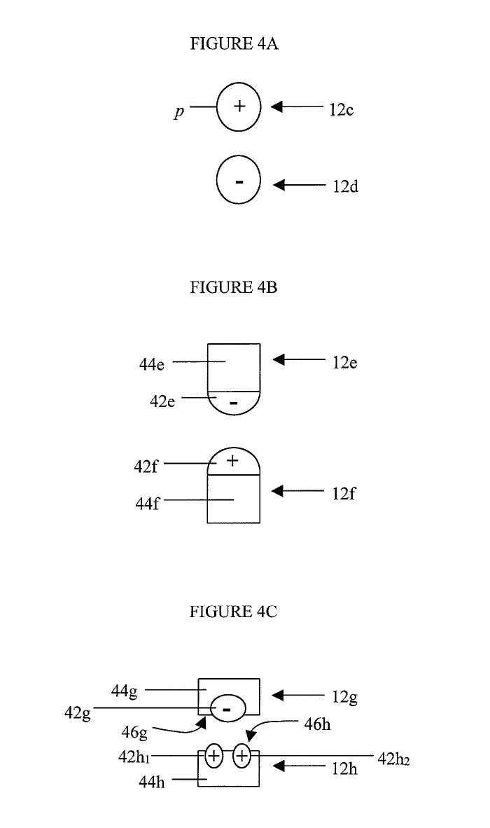

The first and second members 12a, 12b, 12a′, 12b′ can have virtually any shape and size, but preferably each member 12a, 12b, 12a′, 12b′ is substantially elongate and includes a tissue-contacting surface 20a, 20b, 20a′, 20b′ and a tissue-opposing surface 21a, 21b, 21a′, 21b′. Each surface 20a, 20b, 20a′, 20b′, 21a, 21b, 21a′, 21b′ extends along a distal portion 13, 13′ of each member 12a, 12b, 12a′, 12b′. The tissue-contacting surface 20a, 20b, 20a′, 20b′ and tissue-opposing surface 21a, 21b, 21a′, 21b′ of each member 12a, 12b, 12a′, 12b′ defines an outer perimeter p, which can have virtually any shape and size. By way of non-limiting example, the perimeter p can be circular, as shown in FIG. 4A, ovular, square, rectangular, or any other shape. Moreover, the perimeter p can have an irregular shape, as shown in FIGS. 4B-4C. The perimeter p can also vary along the length of the first and second members 12a, 12b, 12a′, 12b′.

Referring back to FIGS. 1-3, the distal portion 13, 13′ of the first and second members 12a, 12b, 12a′, 12b′ can optionally be malleable to allow the members 12a, 12b, 12a′, 12b′ to be formed into a desired shape to conform to the tissue on which it is placed, or to form a desired lesion pattern. In use, the shape of the first and second members 12a, 12b, 12a′, 12b′ is determinative of the shape or pattern of the lesion, or portion of a lesion, to be formed. By way of non-limiting example, the first and second members 12a, 12b, 12a′, 12b′ can be formed to have a curvilinear or circumferential shape (not shown) to allow the members to be positioned, for example, around all of, or a portion of, the pulmonary veins. As a result, the lesion formed in the tissue will have a curvilinear or circumferential shape substantially the same as the shape of the first and second members 12a, 12b, 12a′, 12b′. Various polymer-coated metal rods or wires may be used to impart a suitable degree of malleability to allow the first and second members 12a, 12b, 12a′, 12b′ to be repeatedly and reversibly shaped for successively accessing different cardiac sites. By way of non-limiting example, the first and second members 12a, 12b, 12a′, 12b′ can be formed from parylene coated fully annealed stainless steel. Alternatively, or in addition, the members 12a, 12b, 12a′, 12b′ can be formed from a shape memory material such as, for example, a nickel-titanium alloy, and more particularly, Nitinol.

In an exemplary embodiment, a distal tip 16a, 16b, 16a′, 16b′ of one of the first and second members 12a, 12b, 12a′, 12b′ is adapted to be deployed into or through a tissue surface to position the first and second members 12a, 12b, 12a′, 12b′ on opposed surfaces of tissue. As shown in FIGS. 1-3, the first member 12a, 12a′ includes a distal tissue piercing tip 17, 17′. In an exemplary embodiment, the tissue piercing tip 17, 17′ has a diameter sufficiently small to allow the tip 17, 17′ to puncture a tissue surface without requiring the puncture hole to be sealed after removal of the first member 12a, 12a′. Preferably, the diameter d₁ is equal to or less than 1 mm. In another embodiment, the tip 17, 17′ can have a diameter that is less than about 5 mm. In this case, the puncture would require the surgeon to seal the puncture upon completion of the surgery. The sealed puncture can form part of the lesion pattern.

The tissue piercing tip 17, 17′ can be a solid member, or it can optionally including an inner lumen formed therein and extending through the first member 12a, 12a′. The second member 12b, 12b′ can also, or alternatively, include an inner lumen (not shown) formed therein having a fluid exit port disposed on or near the distal end 13, 13′ of the second member 12b, 12b′. The inner lumen(s) can be provided for introducing irrigation and/or cooling fluid to the ablation site. Irrigation fluid is useful for removing blood from the ablation site, thereby avoiding or reducing the risk of forming blood clots, and cooling fluid is effective to prevent overheating of the tissue or the formation of hot spots during ablation. Irrigating and/or cooling fluids are known in the art and include, for example, saline, lactated Ringer's solution and sterile water. The inner lumen of the first member 12a, 12a′ can also be utilized to determine the penetration depth of the member 12a, 12a′. For example, insertion of the distal tissue piercing tip 17, 17′ through the tissue can cause blood to flow into the inner lumen and thereby cause flashback to occur, thus indicating penetration of the tissue piercing tip 17, 17′ into a blood containing chamber, such as the atrium of the heart. A measurement gauge, or similar device, can then be utilized to insert the remainder of the distal portion 13, 13′ of the first member 12a, 12a′ to a desired depth.

The proximal portion 15, 15′ of each member 12a, 12b, 12a′, 12b′ preferably includes a mechanism for allowing movement of the members 12a, 12b, 12a′, 12b′ relative to each other. The members 12a, 12b, 12a′, 12b′ can be mated together using, for example, one or more rivets, screws, or similar elements which form one or more pivot points between the members 12a, 12b, 12a′, 12b′. In use, one or both of the members 12a, 12b, 12a′, 12b′ can be rotated around the pivot point(s) to move the members 12a, 12b, 12a′, 12b′ between the open and closed positions.

By way of non-limiting example, FIG. 1 illustrates an instrument 10 having two rivets 22a, 22b which mate the first member 12a to the second member 12b. The rivets 22a, 22b form two pivot-points which allow the proximal portion 15 of the first and second members 12a, 12b to be moved in opposed directions, and thereby cause the distal portion 13 of the second member 12b to pivotally rotate with respect to the distal portion 13 of the first member 12a, as shown in FIG. 2.

FIG. 3 illustrates another embodiment of a surgical ablation instrument 10′ wherein a single rivet 22c is disposed through each of the first and second members 12a′, 12b′ to mate the members 12a′, 12b′. As shown, the proximal portion 15′ of each member 12a′, 12b′ can include an angled portion 32a, 32b which is effective to cause the proximal portion 15′ of the members 12a′, 12b′ to cross-over one another. The rivet 22c is disposed through the members 12a′, 12b′ at the cross-section. The angled portions 32a, 23b also allow the distal portion 13′ of each member 12a′, 12b′ to remain parallel to one other. In use, the proximal portion 15′ of each member 12a′, 12b′ can be moved relative to each other to cause the distal portion 13′ of each member 12a′, 12b′ the move between the open and closed positions.

The first and second members 12a, 12b, 12a′, 12b′ can further include an actuating mechanism for effecting movement of the first and second members 12a, 12b, 12a′, 12b′ with respect to each other. A variety of actuating mechanisms can be provided, including both mechanical and electrical actuating mechanisms. In an exemplary embodiment, as shown in FIGS. 1-3, the actuating mechanism is formed from a first grasping element 18a, 18a′ mated to or formed integrally with the first member 12a, 12a′, and a second grasping element 18b, 18b′ mated to or formed integrally with the second member 12b, 12b′. The grasping elements 18a, 18b, 18a′, 18b′ can have a variety of configurations, and can have virtually any shape and size. Preferably, each grasping element 18a, 18b, 18a′, 18b′ is a handle-type member for facilitating gripping of the device 10, 10′. In use, the grasping elements 18a, 18b, 18a′, 18b′ are movable between a first, open position shown in FIGS. 1 and 3 in which the first and second members 12a, 12b, 12a′, 12b′ are in the closed position, and a second, closed position shown in FIG. 2 in which the first and second members 12a, 12b are in the open position.

The first and second members 12a, 12b, 12a′, 12b′ can also optionally include a locking mechanism (not shown) for temporarily locking the first and second members 12a, 12b, 12a′, 12b′ in the closed position while ablative energy is delivered to the tissue. The locking mechanism can be, for example, a clasp or similar device mated to the proximal portion 15, 15′ of each member 12a, 12b, 12a′, 12b′ to retain the members in the closed position. A person having ordinary skill in the art will appreciate that a variety of different mechanisms can be provided to lock the first and second members 12a, 12b, 12a′, 12b′ in one of the open or closed positions.

While FIGS. 1-3 illustrate an instrument 10, 10′ that employs pivotal movement of the members 12a, 12b, 12a′, 12b′ with respect to each other, a person having ordinary skill in the art will appreciate that the surgical ablation instrument 10, 10′ can have a variety of configurations for effecting movement of the members 12a, 12b, 12a′, 12b′ with respect to each other. For example, the first and second members 12a, 12b, 12a′, 12b′ can be slidably movable with respect to each other. In addition, the members 12a, 12b, 12a′, 12b′ may also include one or more mechanisms, such as a suction, compression or adhesion mechanism, to better grip the tissue disposed therebetween and to assure stable contact during the ablation procedure.

As shown in FIG. 3, the ablation instrument 10′ can optionally include a biasing element 26 effective to bias the first and second members 12a, 12b to one of the open or closed positions. The biasing element 26 can have virtually any configuration, but is preferably a spring, or similar device, mated to the first and second grasping elements 18a, 18b′ and/or to the first and second members 12a′, 12b′. In an exemplary embodiment, the grasping elements 18a′, 18b′ are biased to the open position, as shown in FIG. 3, such that the first and second members 12a′, 12b′ are biased to the closed position.

In use, the biasing element 26 applies a force to maintain the first and second members 12a′, 12b′ in one of the open or closed positions. The force should be sufficient to ensure that the first and second members 12a′, 12b′ maintain contact with the tissue disposed therebetween without inflicting unduly high or destructive crushing pressure on the tissue. The force of the biasing element 26 can be overcome by causing the grasping members 18a′, 18b′ to come into contact with each other and thereby move the first and second members 12a′, 12b′ from the closed position, as shown in FIG. 3, to the open position (not shown). Once the first and second members 12a′, 12b′ are in the open position, the instrument 10′ can be manipulated to position the first and second members 12a′, 12b′ as desired. Upon release of the actuating members 18a′, 18b′, the biasing element 26 causes the first and second members 12a′, 12b′ to return to the closed position to grasp tissue disposed therebetween. Ablative energy can then be applied to form a lesion in the grasped tissue.

Ablative energy 50 is applied to the tissue via first and second conductive elements 20a, 20b, 20a′, 20b′ disposed on at least a portion of the first and second members 12a, 12b, 12a′, 12b′. conductive elements 20a, 20b, 20a′, 20b′, e.g. electrodes, are effective to communicate with a source of ablative energy 50. First and second conductor elements 24a, 24b, 24a′, 24b′, e.g., electrically conductive wires, can be provided for separately electrically connecting the conductive elements 20a, 20b, 20a′, 20b′ to the source of ablative energy 50.

The conductive elements 20a, 20b, 20a′, 20b′ can be formed integrally with the first and second members 12a, 12b, 12a′, 12b′, or they can be disposed on a portion thereof. As shown in FIGS. 1-3, the conductive elements 20a, 20b, 20a′, 20b′ are disposed along and form the tissue-contacting portion of each member 12a, 12b, 12a′, 12b′. The length l, width w, and configuration of the conductive elements 20a, 20b, 20a′, 20b′ can vary, but preferably the length l and width w of each conductive element 20a, 20b, 20a′, 20b′ is adapted based on the desired length and width of the lesion to be formed. Preferably the length l of each conductive element 20a, 20b, 20a′, 20b′ is in the range of about 10 mm to 75 mm and the width w is in the range of about 2 mm to 15 mm. The surface area (l×w) of each conductive element 20a, 20b, 20a′ 20b′ can also vary, but preferably the surface area 20a, 20b, 20a′ 20 of one of the conductive elements 12a, 12b, 12a′, 12b′ is greater than the surface area of the oppose conductive element 20a, 20b, 20a′, 20b′.

The conductive elements 20a, 20b, 20a′, 20b′ can be made from any electrically conductive material. Preferred materials include, but are not limited to, conductive composite materials, stainless steel, titanium, platinum, gold, silver, iridium, and alloys thereof.

In use, one of the conductive element 20a, 20b, 20a′, 20b′ serves as an active energy-delivering electrode, while the opposed conductive element is a return electrode which provides a controlled path for the current. The electrosurgical current is thus established through the target tissue, between the energy-delivering electrode and the return electrode. In an exemplary embodiment, the return electrode has a surface area greater than the surface area of the active electrode. Preferably, the conductive surface area of the return electrode is about the same size as the conductive surface area of the energy-delivering electrode.

By way of non-limiting example, FIGS. 4A-4C illustrate a variety of different configurations for the first and second members of the ablation instrument. A person having ordinary skill in the art will appreciate that the first and second members can include any combination of features illustrated and described herein. Moreover, while the first and second members are shown having a similar configuration, a person having ordinary skill in the art will appreciate that the shape, size, and configuration of the first member can vary from the shape, size, and configuration of the second member.

FIG. 4A illustrates a cross-sectional view of first and second members 12c, 12d having opposed polarities. Each member 12c, 12d has a substantially cylindrical shape, and forms a conductive element. In use, one of the members, member 12c for example, is effective to deliver ablative energy through a tissue surface to member 12d, which is effective to receive the ablative energy.

FIG. 4B illustrates another embodiment of first and second members 12e, 12f, each having a tissue-contacting portion 42e, 42f and a tissue-opposing portion 44e, 44f. The tissue-contacting portion 42e, 42f of each member 12e, 12f has a substantially semi-circular shape and forms the conductive element 34e, 34f. The conductive elements 34e, 34f are preferably bipolar. The tissue-opposing portion 44e, 44f of each member 12e, 12f has a substantially block-like or square shape and is mated to the conductive element 34e, 34f to form the first and second members 12e, 12f. In use, when the first and second members 12e, 12f are positioned in the closed position, the tissue-contacting portions 42e, 42f of each member 12e, 12f will be substantially surrounded by the tissue. As a result, ablative energy is essentially transmitted only through the tissue surface disposed therebetween, rather than to surrounding tissue or organs. This configuration is particularly advantageous in that the risk of emboli formation is reduced.

FIG. 4C illustrates yet another embodiment of first and second members 12g, 12h. As shown, the first member 12g includes a conductive tissue-contacting portion 42g and a tissue-opposing portion 44g. The tissue-contacting portion 42g has a substantially circular shape, and is partially surrounded by the tissue-opposing portion 44g, which has a substantially square or block-like shape. The second member 12h includes first and second conductive tissue-contacting portions 42h₁, 42h₂, each of which has a substantially circular shape. The first and second tissue-contacting portions 42h₁, 42h₂ are positioned on opposed sides of the tissue-contacting portion 42g of the first member 12g such that the first and second tissue-contacting portions 42h₁, 42h₂ laterally surround tissue-contacting portion 42g. The second member 12h also includes a tissue-opposing portion 44h which is positioned around the first and second tissue-contacting portions 42h₁, 42h₂. While portions 44g and 44h are described as being tissue-opposing portions, a person having ordinary skill in the art will appreciate that a minor, tissue-facing surface 46g, 46h of each member 12g, 12h may contact the tissue. In use, the conductive tissue-contacting portions 42g, 42h₁, 42h₂ essentially extend into the tissue surface, while the tissue-facing surface 46g, 46h of each member 12g, 12h abuts the tissue surface. Ablative energy is transmitted between the conductive members 42g, 42h₁, 42h₂ to form a lesion in the tissue positioned therebetween.

In an exemplary embodiment, the tissue-opposing portions 44e, 44f, 44g, 44h of each member 12e, 12f, 12g, 12h shown in FIGS. 4B-4C are formed from an insulative coating which prevents ablative energy from extending in directions other than toward the tissue surface. A person having ordinary skill in the art will appreciate that the insulative coating can have a variety of configurations. The insulative coating 34e, 34f, 34g, 34h can be formed from a variety of materials. Suitable materials include ultra high molecular weight polyethylene, polytetra-fluoroethylene (Teflon), nylon, and other biocompatible plastics.

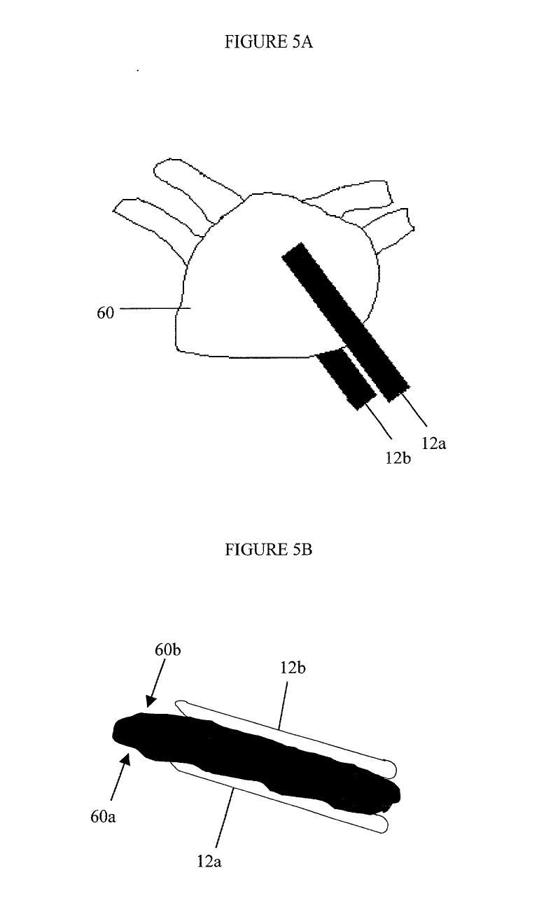

The instrument 10, 10′ according to the present invention can be used on a stopped or beating heart, and either during open-heart surgery or thoracoscopic heart surgery. The procedure can be performed either alone, or in addition to other surgical procedures. FIGS. 5A and 5B illustrate the instrument 10 in use having first and second members 12a, 12b positioned on opposed tissue surfaces 60a, 60b. More particularly, the first member 12a is positioned on an epicardial surface of a heart 60, while the second member 12b is positioned on a endocardial surface of a heart 60.

In use, the first and second members of the instrument 10 are moved to the open position, and the distal tissue piercing tip of the second member is deployed into the tissue surface. Once a substantial portion of the second member 12b is inserted through the tissue, the first and second members 12a, 12b are then moved to the closed position to grasp a portion of tissue disposed therebetween, as shown in FIG. 5B. Ablative energy is then transmitted between the first and second members 12a, 12b to ablate intervening tissue and form a lesion.

The steps of inserting the second member 12b through the tissue and ablating the tissue can be repeated to form a plurality of lesion segments, which together can form a lesion pattern. The lesion pattern is preferably formed around the pulmonary veins of the heart 60 and connected to the mitral valve. The first and second members 12a, 12b can be shaped to fit around the pulmonary veins, or it can be moved to form a lesion having the desired pattern.

One of ordinary skill in the art will appreciate that a variety of electrosurgical generators can be used as the energy source. In one embodiment, the energy source is a radiofrequency (RF) generator that can operate in bipolar and/or monopolar mode. Such a generator should be capable of delivering RF energy having from about 1 to 100 watts of power and a frequency in the range of about 1 KHz to 1 MHz. More preferably, however, the desired frequency is in the range of about 250 KHz to 600 KHz, and the desired wattage is in the range of about 10 to 50 watts.

One of ordinary skill in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A surgical ablation instrument, comprising:
a first member adapted to be positioned adjacent a first tissue surface;
a first conductive element disposed on a portion of the first member and being adapted to communicate with a source of ablative energy;
a second member opposed to and movable relative to the first member, the second member being adapted to be positioned adjacent a second, opposed tissue surface; and
a second conductive element disposed on a portion of the second member and being adapted to communicate with a source of ablative energy;
wherein the first and second conductive elements are effective to transmit ablative energy therebetween.

2. The ablation instrument of claim 1, wherein the first and second members are movable between a first, open position and a second, closed position in which the first member is adjacent to the second member.

3. The ablation instrument of claim 2, further comprising an actuating member mated to the first and second members and effective to selectively move the members between the open and closed positions.

4. The ablation instrument of claim 3, wherein the first and second members are elongate and each member includes a proximal end mated to the actuating member, and a distal portion having the conductive element disposed thereon.

5. The ablation instrument of claim 4, wherein the distal portion of the second member includes a tissue piercing tip adapted to be selectively deployed into tissue.

6. The ablation instrument of claim 4, wherein the first conductive element comprises first and second electrodes extending along the length of the distal portion of the first member and adapted to be positioned adjacent a tissue surface, and the second conductive element comprises a single electrode extending along the length of the distal portion of the second member and adapted to be positioned adjacent an opposed tissue surface between the first and second electrodes of the first member.

7. The ablation instrument of claim 2, further comprising:
a first conductor element extending from the first conductive element and adapted to communicate with the source of ablative energy; and
a second conductor element extending from the second conductive element and adapted to communicate with the source of ablative energy.

8. The ablation instrument of claim 2, wherein one of the first and second conductive elements is an active energy transmitting electrode, and the other one of the first and second conductive elements is a return electrode.

9. The ablation instrument of claim 2, wherein at least one of the first and second members is malleable.

10. The ablation instrument of claim 2, further comprising an insulative coating disposed around a portion of at least one of the first and second members.

11. The ablation instrument of claim 3, wherein the actuating member comprises opposed first and second handles, wherein a force applied to bring the first and second handles in contact with each other causes opening of the first and second members.

12. The ablation instrument of claim 3, wherein the first and second members are biased to the closed position.

13. An ablation instrument, comprising:
first and second conductive members opposed to and movably mated to each other, at least a portion of each conductive member having a tissue-contacting conductive surface, the first and second conductive members being adapted to be disposed on opposed sides of tissue;
first and second conductor elements mated to the first and second conductive members, at least one of the first and second conductor elements being effective to transmit ablative energy to the tissue-contacting conductive surface of at least one of the first and second conductive members.

14. The ablation instrument of claim 13, wherein at least one of the first and second conductive members has a distal tissue piercing tip adapted to be selectively deployed into tissue to position the first and second conductive members on opposed sides of tissue.

15. The ablation instrument of claim 13, wherein the first and second conductive members are pivotally mated to each other, and the instrument further includes an actuating member mated to the first and second conductive members that is effective to move the conductive members between an open position and a closed position.

16. The ablation instrument of claim 13, wherein the tissue-contacting conductive surface of the first conductive member has a surface area greater than a surface area of the tissue-contacting conductive surface of the second conductive member.

17. A method for ablating tissue, comprising:
providing a surgical ablation instrument having
a first conductive member having a tissue-contacting conductive surface and being effective to communicate with a source of ablative energy; and
a second conductive member movably mated to the first conductive member and having a tissue-contacting conductive surface and a distal tissue piercing tip adapted to be selectively deployed through a tissue surface, the second conductive member being effective to communicate with a source of ablative energy;
positioning the first conductive member on a first surface of a target tissue;
deploying the distal tissue piercing tip of the second conductive member through the tissue surface to position the second conductive member on a second, opposed tissue surface adjacent the first conductive member; and
communicating ablative energy through the tissue between the first and second conductive members to form a lesion segment in the target tissue.

18. The method of claim 17, wherein the first and second conductive members each include an electrical conductor attached to the conductive member and the source of ablative energy.

19. The method of claim 17, wherein the tissue is cardiac tissue and the first conductive member is positioned on an epicardial surface of cardiac tissue, and the second conductive member is deployed to position the conductive member adjacent an endocardial surface of cardiac tissue.
